(19) United States
(12) Patent Application Publication
(10) Pub. No.: US 2018/0282260 A1
(43) Pub. Date: Oct. 4, 2018

(54) 1-AMINOCYCLOPROPANE-1-CARBOXYLIC ACID POLYMORPHS

(71) Applicant: Valent BioSciences LLC, Libertyville, IL (US)

(72) Inventors: Mitsunobu Kawamura, Tokyo (JP); Daniel F. Heiman, Libertyville, IL (US); Kosuke Takebayashi, Tokyo (JP); Gary T. Wang, Libertyville, IL (US)

(21) Appl. No.: 15/940,364

(22) Filed: Mar. 29, 2018

Related U.S. Application Data
(60) Provisional application No. 62/479,553, filed on Mar. 31, 2017.

(51) Int. Cl. C07C 229/48; C07C 227/42; A01N 53/00

(52) U.S. Cl. CPC C07C 229/48; C07C 227/42; C07C 2601/02; C07B 2200/13; A01N 53/00

(57) ABSTRACT

The present invention is directed to 1-aminocyclopropane-1-carboxylic acid polymorphs and agricultural compositions thereof.

### FIELD OF THE INVENTION

The present invention relates to novel 1-aminocyclopropane-1carboxylic acid polymorphs and agricultural compositions thereof.

### BACKGROUND OF THE INVENTION

1-aminocyclopropane-1-carboxylic acid (“ACC”) is a compound having the following chemical structure:

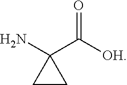

ACC is an ethylene precursor. Ethylene has been shown to be involved in several plant functions including stress responses, fruit set, leaf abscission and anthesis. ACC has been described in several polymorphic forms. One such anhydrous form of ACC is readily available from suppliers such as Sigma-Aldrich and Chem-Impex International, Inc.

Polymorphism is the occurrence of different crystal forms. A single compound, like ACC, may give rise to an assortment of crystalline forms having distinct crystal structures and physical characteristics giving distinct results such as x-ray diffraction patterns or solubilities in various solvents. One crystalline form may give rise to thermal behavior different from that of another crystalline form. Thermal behavior can be measured in the laboratory by such techniques as thermogravimetry and differential thermal analysis, which have been used to distinguish polymorphic forms.

The difference in the physical properties of different crystalline forms results from the orientation and intermolecular interactions of adjacent molecules. Accordingly, polymorphs are distinct forms sharing the same molecular formula yet having distinct advantageous physical properties compared to other crystalline forms of the same compound.

The discovery of new polymorphs of known compounds such as ACC can give rise to new uses or improved function in existing uses. Thus, there exists a need in the art for new polymorphic forms of ACC.

### SUMMARY OF THE INVENTION

In one aspect, the present invention is directed to 1-aminocyclopropane-1-carboxylic acid (“ACC”) trihydrate.

In another aspect, the present invention is directed to an ACC trihydrate characterized by an x-ray powder diffraction having peaks at about 16.1, 17.3, 22.2, 24.8, 29.8 and 30.4 2 Θ degrees.

In another aspect, the present invention is directed to an ACC trihydrate characterized by an x-ray powder diffraction pattern as depicted in FIG. 2.

In another aspect, the present invention is directed to ACC trihydrate produced by the process comprising the steps of;
mixing 1 part water with 1.75 parts of a 1-aminocyclopropane-1-carboxylic acid anhydrate characterized by an x-ray powder diffraction pattern as depicted in FIG. 1 to produce a mixture; and
heating the mixture to remove the water

In another embodiment, the present invention is directed to an ACC anhydrate characterized by an x-ray powder diffraction pattern as depicted in FIG. 3.

In another embodiment, the present invention is directed an ACC anhydrate characterized by an x-ray powder diffraction having peaks at about 9.7, 15.8, 16.7, 19.4, 20.7, 22.3, 24.9, 26.8 and 37.0 2 Θ degrees.

In another aspect, the present invention is directed to an ACC anhydrate produced by the process comprising the steps of
adding 1 part of 1-aminocyclopropane-1-carboxylic acid trihydrate to 4 parts of toluene to produce a slurry; and
drying the slurry under a nitrogen gas stream for 5 hours at 25° C.

In another aspect, the present invention is directed to an ACC anhydrate characterized by a thermogravimetry and differential thermal analysis substantially as depicted in FIG. 5.

In another aspect, the present invention is directed to an agricultural composition comprising the ACC trihydrate of the present invention and one or more agricultural excipients,

In another aspect, the present invention is directed to an agricultural composition comprising the ACC anhydrate of the present invention and one or more agricultural excipients.

### BRIEF DESCRIPTION OF THE DRAWINGS

FIG. 1. X-ray powder diffraction (“XRD”) of a known 1-aminocyclopropane-1-carboxylic acid anhydrate from Chem-Impex International, Inc.

FIG. 2. XRD of 1-aminocyclopropane-1-carboxylic acid trihydrate.

FIG. 3. XRD of 1-aminocyclopropane-1-carboxylic acid anhydrate of the present invention.

FIG. 4. Thermogravimetry and differential thermal analysis (“TG-DTA”) of a known 1-aminocyclopropane-1-carboxylic acid anhydrate from Chem-Impex International, Inc.

FIG. 5. TG-DTA of 1-aminocyclopropane-1-carboxylic acid anhydrate of the present invention.

### DETAILED DESCRIPTION OF THE INVENTION

The present invention is directed to novel 1-aminocyclopropane-1-carboxylic acid (“ACC”) polymorphs. The ACC trihydrate polymorph of the present invention has been discovered to be the most stable crystal form of ACC under high humidity. Surprisingly, a novel ACC anhydrate polymorph created from drying the ACC trihydrate of the present invention under particular conditions has a different crystal structure than currently available ACC anhydrates.

In one embodiment, the present invention is directed to ACC trihydrate.

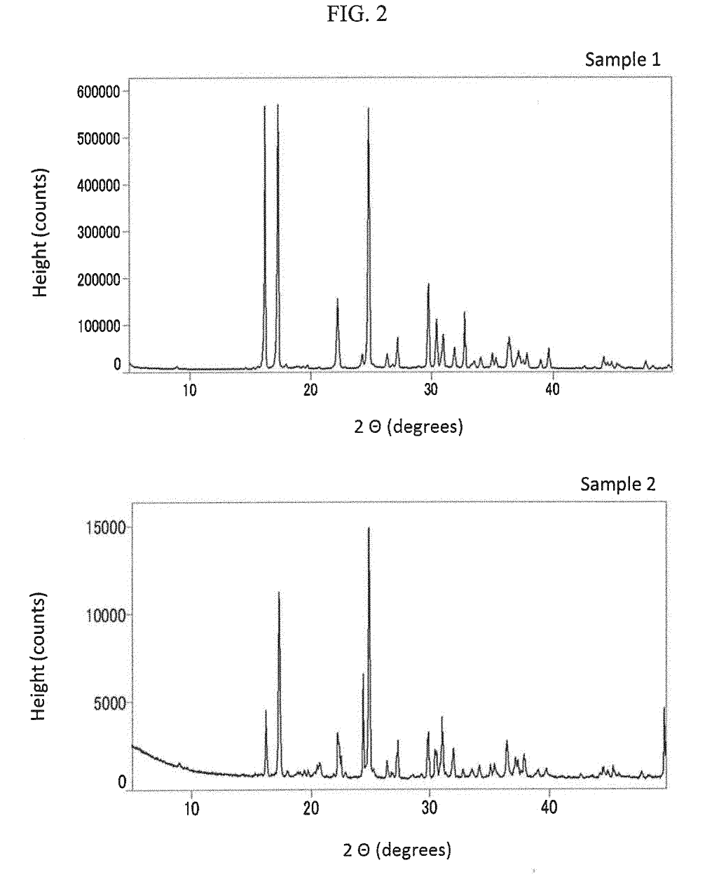

In another embodiment, the present invention is directed an ACC trihydrate characterized by an x-ray powder diffraction pattern as depicted in FIG. 2.

In another embodiment, the present invention is directed to an ACC trihydrate characterized by an x-ray powder diffraction having peaks at about 16.1, 17.3, 22.2, 24.8, 29.8 and 30.4 2 Θ degrees.

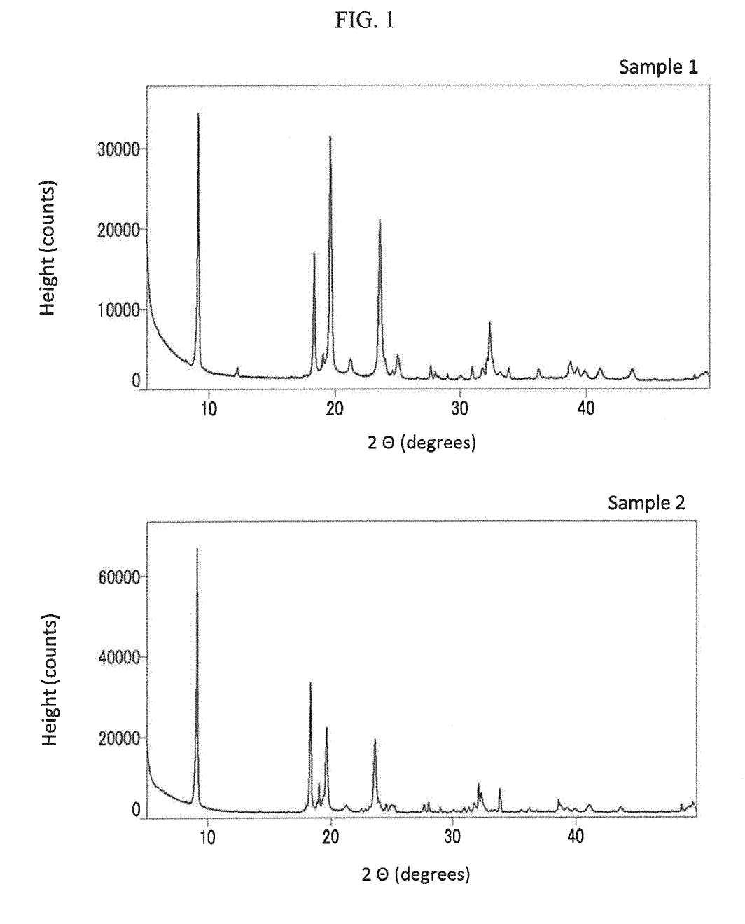

In another embodiment, the present invention is directed to an ACC trihydrate produced by the process comprising the steps of:
mixing 1 part water with 1.75 parts of a 1-aminocyclopropane-1-carboxylic acid anhydrate characterized by an x-ray powder diffraction pattern as depicted in FIG. 1 to produce a mixture; and
heating the mixture to remove the water.

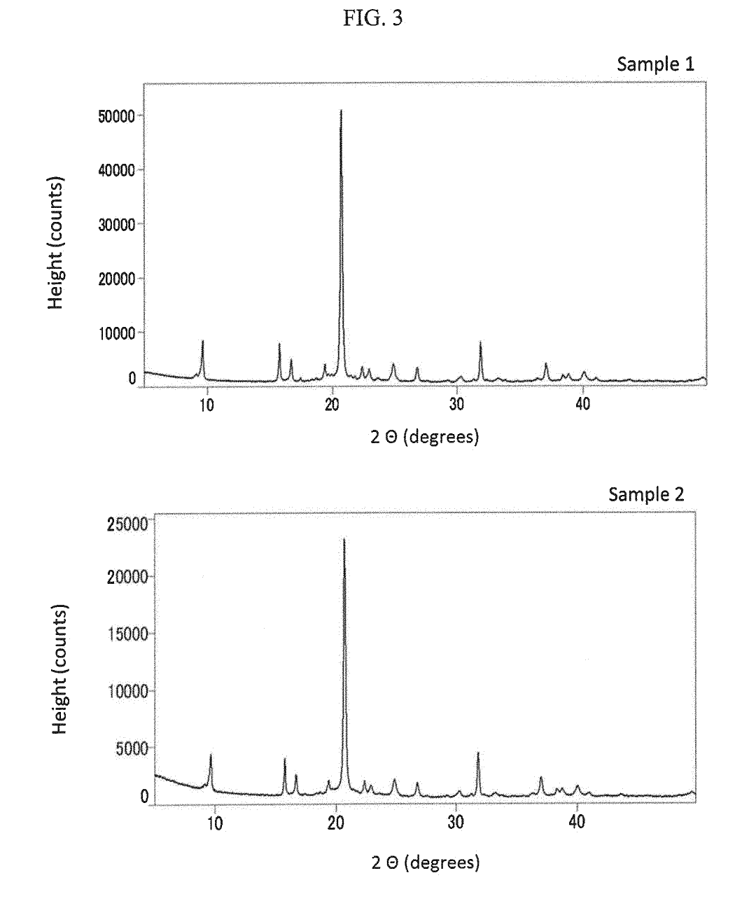

In another embodiment, the present invention is directed to an ACC anhydrate characterized by an x-ray powder diffraction pattern as depicted in FIG. 3.

In another embodiment, the present invention is directed to an ACC anhydrate characterized by a thermogravimetry and differential thermal analysis substantially as depicted in FIG. 5.

In another embodiment, the present invention is directed to an ACC anhydrate characterized by an x-ray powder diffraction having peaks at about 9.7, 15.8, 16,7, 19.4, 20,7, 22.3, 24.9, 26,8 and 37.0 2 Θ degrees.

In another embodiment, the present invention is directed to 1-aminocyclopropane-1-carboxylic acid anhydrate produced by the process comprising the steps of:
adding 1 part of 1-aminocyclopropane-1-carboxylic acid trihydrate to 4 parts of toluene to produce a slurry; and
drying the slurry under a nitrogen gas stream for 5 hours at 25° C.

In another embodiment, the present invention is directed to an agricultural composition comprising the ACC trihydrate of the present invention and one or more agricultural excipients.

In another embodiment, the present invention is directed to an agricultural composition comprising the ACC anhydrate of the present invention and one or more agricultural excipients.

As used herein, the term “about” when used in conjunction with peak values denotes the peak value ±0.2 degrees 2 Θ.

Agricultural excipients include, but are not limited to, surface active agents, dispersants, wetter-spreaders, stickers, penetrants, binders, polymers, pH regulators, drift control agents, UV protectants, colorants, microencapsulating agents, sugars, starches, free-flow agents, clays, nutrients and humectants.

For a clearer understanding of the invention, Examples are provided below. These are merely illustrations and are not to be understood as limiting the scope of the invention in any way, indeed, various modifications of the invention in addition to those shown and described herein will become apparent to those skilled in the art from the following examples and foregoing description. Such modifications are also intended to fall within the scope of the appended claims.

### EXAMPLES

### Example 1

### Process of Preparation

### Preparation of 1-aminocyclopropane-1-carboxylic acid trihydrate

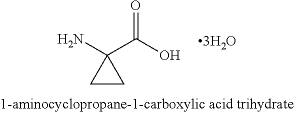

To 1-aminocyclopropane-1-carboxylic acid (“ACC”) anhydrate powder (3.0 g, purchased from Chem-Impex International, Inc.) was added water (1.71 g) and mixed in a mortar at 25° C., Heat was evolved upon mixing to give a powder (4.65 g). Water content was measured by Karl. Fischer Method and found to be 33.2%. (95% of theoretical value),

Preparation of 1-aminocyclopropane-1-carboxylic acid anhydrate

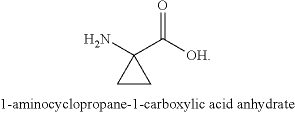

A solution of 1-aminocyclopropane-1-carboxylic acid trihydrate as synthesized above (5 g), was added toluene (20 g). The resulting toluene slurry was filtered and resulting wet cake was dried under a nitrogen gas stream for 5 hours at 25° C. to obtain 94% pure ACC anhydrate as a white powder (3.1 g). Water content was measured by Karl Fisher Method and found to be 0.50%.

### Example 2

### X-ray Powder Diffractions

2 x-ray powder diffractions were performed on each of the ACC anhydrate from Chem-Impex International, Inc., the ACC trihydrate of the present invention and the ACC anhydrate of the present invention. X-ray powder diffraction was performed on a Rigaku SmartLab® powder diffractometer. Prior to analysis the samples were gently ground by means of mortar and pestle in order to obtain a fine powder.

TABLE 1
X-ray powder diffraction of ACC anhydrate
from Chem-Impex International, Inc. (Sample 1)

| Peak No. | 2θ (degrees) | Height (counts) | Relative Height (%) |
|---|---|---|---|
| 1 | 9.1491 | 21608.3 | 22.6 |
| 2 | 12.2413 | 675.84 | 0.7 |
| 3 | 18.3331 | 10758.02 | 11.3 |
| 4 | 19.0216 | 1381.31 | 1.4 |
| 5 | 19.6374 | 21165.78 | 22.2 |
| 6 | 21.2329 | 1375.73 | 1.4 |
| 7 | 23.5918 | 13651.64 | 14.3 |
| 8 | 23.9818 | 909.25 | 1.0 |
| 9 | 24.5833 | 521.55 | 0.5 |
| 10 | 25.0085 | 1875.67 | 2.0 |
| 11 | 27.5623 | 37.14 | 0.0 |
| 12 | 27.6679 | 1308.82 | 1.4 |
| 13 | 28.0211 | 592.05 | 0.6 |
| 14 | 29.0018 | 530.99 | 0.6 |
| 15 | 30.085 | 313.58 | 0.3 |
| 16 | 30.9517 | 1487.99 | 1.6 |
| 17 | 31.7982 | 959.12 | 1.0 |
| 18 | 32.1114 | 2061.16 | 2.2 |
| 19 | 32.3194 | 5221.9 | 5.5 |
| 20 | 33.1279 | 364.91 | 0.4 |
| 21 | 33.8614 | 1113.59 | 1.2 |
| 22 | 36.1961 | 1018.25 | 1.1 |
| 23 | 38.6143 | 1290.45 | 1.4 |
| 24 | 38.7736 | 523.29 | 0.5 |
| 25 | 39.256 | 851.43 | 0.9 |
| 26 | 39.8709 | 666.4 | 0.7 |
| 27 | 41.0801 | 843.81 | 0.9 |
| 28 | 43.6458 | 938.59 | 1.0 |
| 29 | 45.4843 | 73.86 | 0.1 |
| 30 | 48.7807 | 479.54 | 0.5 |
| 31 | 49.2854 | 328 | 0.3 |
| 32 | 49.6955 | 561.61 | 0.6 |
| Total | | 95489.57 | 100.0 |

TABLE 2
X-ray powder diffraction of ACC anhydrate
from Chem-Impex International, Inc. (Sample 2)

| Peak No. | 2θ (degrees) | Height (counts) | Relative Height (%) |
|---|---|---|---|
| 1 | 9.1215 | 46398.24 | 31.5 |
| 2 | 14.2943 | 96.07 | 0.1 |
| 3 | 18.1731 | 1264 | 0.9 |
| 4 | 18.3348 | 21573.38 | 14.7 |
| 5 | 18.8404 | 1068.77 | 0.7 |
| 6 | 19.0219 | 4482.12 | 3.0 |
| 7 | 19.3656 | 2163.83 | 1.5 |
| 8 | 19.6367 | 13876.47 | 9.4 |
| 9 | 21.2581 | 877.23 | 0.6 |
| 10 | 22.4713 | 469.57 | 0.3 |
| 11 | 22.7986 | 311.07 | 0.2 |
| 12 | 23.1304 | 543.74 | 0.4 |
| 13 | 23.5909 | 12215.97 | 8.3 |
| 14 | 23.9868 | 1253.08 | 0.9 |
| 15 | 24.5041 | 1326.84 | 0.9 |
| 16 | 24.8519 | 336.09 | 0.2 |
| 17 | 25.0013 | 1137.25 | 0.8 |
| 18 | 25.1825 | 1020.38 | 0.7 |
| 19 | 27.6378 | 1709.87 | 1.2 |
| 20 | 28.0176 | 2033.64 | 1.4 |
| 21 | 28.9646 | 1163.83 | 0.8 |
| 22 | 30.04 | 349.85 | 0.2 |
| 23 | 30.4104 | 238 | 0.2 |
| 24 | 30.9313 | 808.78 | 0.5 |
| 25 | 31.2971 | 945.32 | 0.6 |
| 26 | 31.7711 | 1600.72 | 1.1 |
| 27 | 32.1131 | 6640.08 | 4.5 |
| 28 | 32.2984 | 3556.08 | 2.4 |
| 29 | 33.4274 | 240.1 | 0.2 |
| 30 | 33.8282 | 5500.45 | 3.7 |
| 31 | 35.5322 | 333.72 | 0.2 |
| 32 | 36.1836 | 766.07 | 0.5 |
| 33 | 36.543 | 147.03 | 0.1 |
| 34 | 36.7804 | 340.5 | 0.2 |
| 35 | 38.5866 | 2377.95 | 1.6 |
| 36 | 39.3232 | 584.41 | 0.4 |
| 37 | 39.91 | 554.46 | 0.4 |
| 38 | 41.0809 | 1123.26 | 0.8 |
| 39 | 43.6382 | 824.57 | 0.6 |
| 40 | 46.9256 | 186.05 | 0.1 |
| 41 | 47.9871 | 294.72 | 0.2 |
| 42 | 48.7554 | 1836.56 | 1.2 |
| 43 | 49.1632 | 368.62 | 0.3 |
| 44 | 49.4143 | 642.48 | 0.4 |
| 45 | 49.6881 | 1590.07 | 1.1 |
| Total | | 147171.29 | 100.0 |

TABLE 3
X-ray powder diffraction of ACC trihydrate
of the present invention. (Sample 1)

| Peak No. | 2θ (degrees) | Height (counts) | Relative Height (%) |
|---|---|---|---|
| 1 | 8.9604 | 3738.44 | 0.2 |
| 2 | 9.6033 | 234.52 | 0.0 |
| 3 | 12.2816 | 521.36 | 0.0 |
| 4 | 13.5272 | 949.46 | 0.0 |
| 5 | 14.2147 | 847.22 | 0.0 |
| 6 | 14.6293 | 3417.28 | 0.1 |
| 7 | 15.2552 | 1594.37 | 0.1 |
| 8 | 15.5812 | 2388.14 | 0.1 |
| 9 | 16.1073 | 52812.81 | 2.2 |
| 10 | 16.2059 | 370885.45 | 15.3 |
| 11 | 17.0823 | 17506.19 | 0.7 |
| 12 | 17.3074 | 383253.59 | 15.8 |
| 13 | 17.9267 | 5481.23 | 0.2 |
| 14 | 19.2928 | 1884.48 | 0.1 |
| 15 | 19.6949 | 4527.22 | 0.2 |
| 16 | 20.6458 | 1593.27 | 0.1 |
| 17 | 21.9269 | 4234.32 | 0.2 |
| 18 | 22.1943 | 95345.05 | 3.9 |
| 19 | 22.3137 | 34715.47 | 1.4 |
| 20 | 22.8246 | 1276.65 | 0.1 |
| 21 | 24.2631 | 19306.48 | 0.8 |
| 22 | 24.8034 | 299830.71 | 12.3 |
| 23 | 24.8428 | 241085.05 | 9.9 |
| 24 | 25.0834 | 3583.71 | 0.1 |
| 25 | 26.2427 | 2230.69 | 0.1 |
| 26 | 26.3415 | 19829.13 | 0.8 |
| 27 | 26.7804 | 4331.79 | 0.2 |
| 28 | 27.2074 | 46934.51 | 1.9 |
| 29 | 28.9824 | 1560.04 | 0.1 |
| 30 | 29.7823 | 137153.58 | 5.6 |
| 31 | 30.4333 | 78532.32 | 3.2 |
| 32 | 30.8244 | 16054.34 | 0.7 |
| 33 | 30.9839 | 45917.49 | 1.9 |
| 34 | 31.9166 | 32422.07 | 1.3 |
| 35 | 32.1449 | 1761.13 | 0.1 |
| 36 | 32.6564 | 16412.39 | 0.7 |
| 37 | 32.7484 | 87632.95 | 3.6 |
| 38 | 33.3213 | 5009.9 | 0.2 |
| 39 | 33.5394 | 10090.17 | 0.4 |
| 40 | 34.045 | 15652.98 | 0.6 |
| 41 | 35.026 | 22978.74 | 0.9 |
| 42 | 35.32 | 15198.57 | 0.6 |
| 43 | 36.3123 | 24534.1 | 1.0 |
| 44 | 36.3907 | 34348.12 | 1.4 |
| 45 | 36.5218 | 26026.17 | 1.1 |
| 46 | 37.0031 | 9617.36 | 0.4 |
| 47 | 37.1835 | 23251.48 | 1.0 |
| 48 | 37.3352 | 10716.98 | 0.4 |
| 49 | 37.5642 | 12641.27 | 0.5 |
| 50 | 37.8696 | 24831.2 | 1.0 |
| 51 | 39.0018 | 13731.92 | 0.6 |
| 52 | 39.5955 | 32948.52 | 1.4 |
| 53 | 40.7182 | 468.09 | 0.0 |
| 54 | 41.9675 | 1236.22 | 0.1 |
| 55 | 42.2088 | 740.19 | 0.0 |
| 56 | 42.607 | 4382.88 | 0.2 |
| 57 | 43.4574 | 2271.82 | 0.1 |
| 58 | 44.1207 | 7504.19 | 0.3 |
| 59 | 44.2197 | 19545.4 | 0.8 |
| 60 | 44.5521 | 10108.89 | 0.4 |
| 61 | 44.7763 | 4208.83 | 0.2 |
| 62 | 44.8797 | 11046.48 | 0.5 |
| 63 | 45.3275 | 8964.65 | 0.4 |
| 64 | 45.563 | 5233.25 | 0.2 |
| 65 | 45.8382 | 1527.41 | 0.1 |
| 66 | 46.3305 | 1664.15 | 0.1 |
| 67 | 46.55 | 1489.85 | 0.1 |
| 68 | 47.1497 | 792.17 | 0.0 |
| 69 | 47.7526 | 13733.37 | 0.6 |
| 70 | 48.3369 | 3797.24 | 0.2 |
| 71 | 48.9781 | 1172.32 | 0.0 |
| 72 | 49.3519 | 1087.95 | 0.0 |
| 73 | 49.7232 | 5623.84 | 0.2 |
| Total | | 2429961.57 | 100.0 |

TABLE 4
X-ray powder diffraction of ACC trihydrate
of the present invention. (Sample 2)

| Peak No. | 2θ (degrees) | Height (counts) | Relative Height (%) |
|---|---|---|---|
| 1 | 16.1588 | 2190.51 | 3.4 |
| 2 | 17.2787 | 7377.58 | 11.5 |
| 3 | 17.979 | 190.29 | 0.3 |
| 4 | 19.4249 | 163.01 | 0.3 |
| 5 | 19.7197 | 303.73 | 0.5 |
| 6 | 20.4678 | 329.7 | 0.5 |
| 7 | 20.6985 | 526.75 | 0.8 |
| 8 | 21.8551 | 141.66 | 0.2 |
| 9 | 22.1856 | 2290.83 | 3.6 |
| 10 | 22.308 | 1377.59 | 2.1 |
| 11 | 22.4951 | 960.12 | 1.5 |
| 12 | 22.8398 | 200.85 | 0.3 |
| 13 | 24.3697 | 5499.34 | 8.6 |
| 14 | 24.8412 | 12214.03 | 19.0 |
| 15 | 24.9379 | 3299.03 | 5.1 |
| 16 | 25.2102 | 278.53 | 0.4 |
| 17 | 26.3696 | 810.03 | 1.3 |
| 18 | 26.7643 | 204.86 | 0.3 |
| 19 | 27.1885 | 1001.43 | 1.6 |
| 20 | 27.3087 | 1360.15 | 2.1 |
| 21 | 28.5477 | 96.2 | 0.1 |
| 22 | 29.2781 | 133.25 | 0.2 |
| 23 | 29.8823 | 1900.73 | 3.0 |
| 24 | 30.4342 | 1362.57 | 2.1 |
| 25 | 30.5926 | 980.24 | 1.5 |
| 26 | 31.0446 | 2719.93 | 4.2 |
| 27 | 31.1304 | 1342.45 | 2.1 |
| 28 | 31.3426 | 165.32 | 0.3 |
| 29 | 32.0043 | 1106.78 | 1.7 |
| 30 | 32.7813 | 344.4 | 0.5 |
| 31 | 33.5395 | 235.81 | 0.4 |
| 32 | 34.1181 | 466.54 | 0.7 |
| 33 | 35.0782 | 587.86 | 0.9 |
| 34 | 35.3919 | 371.96 | 0.6 |
| 35 | 36.4062 | 1348.28 | 2.1 |
| 36 | 36.4664 | 1197.52 | 1.9 |
| 37 | 37.1677 | 699.2 | 1.1 |
| 38 | 37.3976 | 554.22 | 0.9 |
| 39 | 37.8844 | 1011.28 | 1.6 |
| 40 | 38.7947 | 118.83 | 0.2 |
| 41 | 39.0323 | 308.76 | 0.5 |
| 42 | 39.6924 | 338.3 | 0.5 |
| 43 | 42.6568 | 198.14 | 0.3 |
| 44 | 44.3277 | 261.06 | 0.4 |
| 45 | 44.5069 | 613.37 | 1.0 |
| 46 | 44.957 | 324.27 | 0.5 |
| 47 | 45.3996 | 543.46 | 0.8 |
| 48 | 47.8372 | 301.68 | 0.5 |
| 49 | 49.8333 | 3871.74 | 6.0 |
| Total | | 64224.17 | 100.0 |

TABLE 5

| | | | |
|---|---|---|---|
| Peak No. | 2θ (degrees) | Height (counts) | Relative Height (%) |

X-ray powder diffraction of ACC anhydrate
of the present invention. (Sample 1)

| | | | |
|---|---|---|---|
| 1 | 9.1403 | 485.08 | 0.7 |
| 2 | 9.6592 | 4979.65 | 7.1 |
| 3 | 15.7714 | 4948.33 | 7.1 |
| 4 | 16.7218 | 2922.01 | 4.2 |
| 5 | 17.4203 | 369.6 | 0.5 |
| 6 | 19.4074 | 1855.43 | 2.6 |
| 7 | 19.6192 | 379.28 | 0.5 |
| 8 | 20.7092 | 34531.21 | 49.3 |
| 9 | 22.3464 | 1414.87 | 2.0 |
| 10 | 22.9175 | 1129.77 | 1.6 |
| 11 | 24.9065 | 2052.39 | 2.9 |
| 12 | 26.7875 | 1856.96 | 2.7 |
| 13 | 27.6327 | 128.36 | 0.2 |
| 14 | 29.2534 | 196.69 | 0.3 |
| 15 | 30.2641 | 589.16 | 0.8 |
| 16 | 31.2248 | 162.84 | 0.2 |
| 17 | 31.851 | 5838.78 | 8.3 |
| 18 | 33.2578 | 398.98 | 0.6 |
| 19 | 36.3069 | 294.81 | 0.4 |
| 20 | 37.0207 | 2336.14 | 3.3 |
| 21 | 38.352 | 708.4 | 1.0 |
| 22 | 38.7927 | 834.32 | 1.2 |
| 23 | 40.0058 | 1052.19 | 1.5 |
| 24 | 41.0247 | 353.13 | 0.5 |
| 25 | 43.6445 | 248.69 | 0.4 |
| Total | | 70067.07 | 100.0 |

X-ray powder diffraction of ACC anhydrate
of the present invention. (Sample 2)

| | | | |
|---|---|---|---|
| 1 | 9.6485 | 2140.99 | 6.7 |
| 2 | 15.7369 | 2505.43 | 7.8 |
| 3 | 16.6878 | 1247.6 | 3.9 |
| 4 | 19.3726 | 652.15 | 2.0 |
| 5 | 19.5076 | 176.11 | 0.5 |
| 6 | 20.69 | 15437.69 | 48.1 |
| 7 | 22.3472 | 747.92 | 2.3 |
| 8 | 22.9032 | 534.18 | 1.7 |
| 9 | 23.5907 | 78.01 | 0.2 |
| 10 | 24.8003 | 889.35 | 2.8 |
| 11 | 26.767 | 766.48 | 2.4 |
| 12 | 30.2843 | 314.08 | 1.0 |
| 13 | 31.2852 | 151.28 | 0.5 |
| 14 | 31.8333 | 3077.56 | 9.6 |
| 15 | 33.1428 | 182.35 | 0.6 |
| 16 | 36.3252 | 154.06 | 0.5 |
| 17 | 37.0374 | 1239.72 | 3.9 |
| 18 | 38.3332 | 441.21 | 1.4 |
| 19 | 38.7605 | 470.87 | 1.5 |
| 20 | 39.984 | 571.28 | 1.8 |
| 21 | 41.0399 | 199.54 | 0.6 |
| 22 | 43.6715 | 124.38 | 0.4 |
| Total | | 32102.24 | 100.0 |

The ACC trihydrate prepared by the process in Example 1, above, gave peaks as listed in Tables 3 and 4 and as seen in FIG. 2. The ACC anhydrate prepared by the process Example 1, above, gave peaks as listed in Table 5 and 6 and as seen in FIG. 3.

### Example 3

### Thermogravimetry and Differential Thermal Analysis

Thermogravimetry and differential thermal analysis was performed on each of the ACC anhydrate from Chem-Impex International, Inc. and the ACC anhydrate of the present invention. Therrnogravimetry and differential thermal analysis was performed on a Bruker TG-DTA2000SR thermogravimetry/differential analyzer heating the sample to 30 to 550° C. at a rate of 5° C./min. Prior to analysis the samples were gently ground by means of mortar and pestle in order to obtain a fine powder.

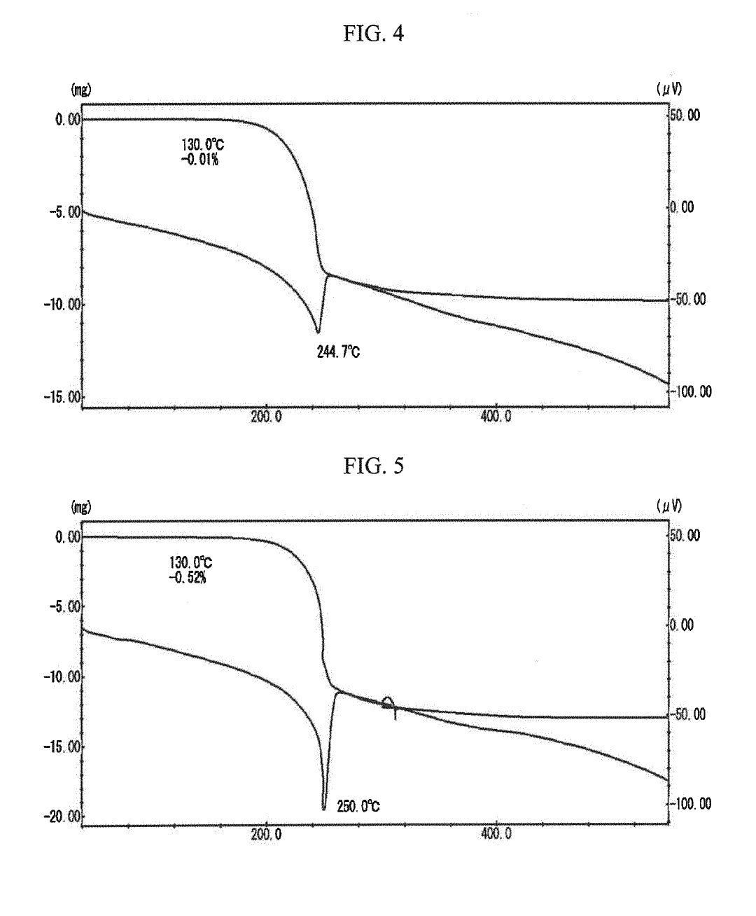

As seen when comparing FIGS. 4 and 5, the ACC anhydrate prepared by the process of Example 1, above, had a much greater change in mass around 250° C. than the ACC anhydrate from Chem-Impex International, Inc.

### Example 4

### Cotton Cotyledon Bioefficacy Assay

### Method

The ACC anhydrate from Chem-Impex International, Inc., an ACC anhydrate prepared by Inogent Laboratories of India and the ACC trihydrate of the present invention were each dissolved with water to 500 ppm w!w of ACC and applied separately to individual sets of 10-day old cotton cotyledons by a hand sprayer in 4 separate trials. 48 hours after application the cotyledons were removed and incubated in sealed vials for 4 to 7 hours. Following incubation, the cotyledons were measured for ethylene gas production by gas chromatography.

TABLE 6
Ethylene production in cotton cotyledons
following application of 500 ppm ACC

| | | Chem-Impex | |
|---|---|---|---|
| | Inogent Anhydrate | Anhydrate | Trihydrate |
| Trial | (nL C₂H₄/g/hr) | (nL C₂H₄/g/hr) | (nL C₂H₄/g/hr) |
| 1 | 25.00 | 28.14 | 15.06 |
| 2 | 20.33 | 23.18 | 22.04 |
| 3 | 17.53 | | 36.30 |
| 4 | 17.43 | | 17.46 |
| Average | 20.07 | 25.66 | 22.71 |
| Standard Error | 1.77 | | 4.75 |

### Results

As seen in Table 6, the ACC trihydrate of the present invention produces a similar ethylene production as readily available anhydrous forms.

What is claimed is:

1. A trihydrate form of 1-aminocyclopropane-1-carboxylic acid.

2. The 1-aminocyclopropane-1-carboxylic acid trihydrate of claim 1 characterized by data consisting of an x-ray powder diffraction having peaks at about 16.1, 17.3, 22.2, 24.8, 29.8 and 30.4 2 Θ degrees.

3. The 1-aminocyclopropane-1-carboxylic acid trihydrate of claim 1 characterized by an x-ray powder diffraction pattern as depicted in FIG. 2.

4. The 1-aminocyclopropane-1-carboxylic acid trihydrate of claim 1 produced by the process comprising the steps of:
mixing 1 part water with 1.75 parts of a 1-aminocyclopropane-1-carboxylic acid anhydrate characterized by an x-ray powder diffraction pattern as depicted in FIG. 1 to produce a mixture.

5. An agricultural composition comprising the 1-aminocyclopropane-1-carboxylic acid trihydrate of claim 1 and one or more agricultural excipients.

6. A 1-aminocyclopropane-1-carboxylic acid anhydrate characterized by an x-ray powder diffraction having peaks at about 9.7, 15.8, 16.7, 19.4, 20.7, 22.3, 24.9, 26.8 and 37.0 2 Θ degrees.

7. The 1-aminocyclopropane-1-carboxylic acid anhydrate of claim 6 characterized by an x-ray powder diffraction pattern as depicted in FIG. 3.

8. An agricultural composition comprising the 1-aminocyclopropane-1-carboxylic acid trihydrate of claim 6 and one or more agricultural excipients.

9. A 1-aminocyclopropane-1-carboxylic acid anhydrate characterized by a thermogravimetry and differential thermal analysis substantially as depicted in FIG. 5.

10. A 1-aminocyclopropane-1-carboxylic acid anhydrate produced by the process comprising the steps of: adding 1 part of 1-aminocyclopropane-1-carboxylic acid trihydrate to 4 parts of toluene to produce a slurry; and filter the slurry; and
drying the resulting wet cake under a nitrogen gas stream for 5 hours at 25 ‘C.
